(19) United States
(12) Patent Application Publication
(10) Pub. No.: US 2023/0034383 A1
(43) Pub. Date: Feb. 2, 2023

(54) METHOD FOR REDUCING SKIN SENSITIZATION OF AN ASENAPINE-CONTAINING PATCH

(71) Applicant: HISAMITSU PHARMACEUTICAL CO., INC., Tosu-shi (JP)

(72) Inventors: Yuka TAKAGI, Tsukuba-shi (JP); Kazuya ISHIDA, Tsukuba-shi (JP); Ryoma TAMURA, Tsukuba-shi (JP); Takao KUROKAWA, Tsukuba-shi (JP); Yasunari MICHINAKA, Tsukuba-shi (JP)

(73) Assignee: HISAMITSU PHARMACEUTICAL CO., INC., Tosu-shi (JP)

(21) Appl. No.: 17/381,294

(22) Filed: Jul. 21, 2021

(51) Int. Cl. A61K 31/407; A61K 9/70; A61K 47/06

(52) U.S. Cl. CPC A61K 31/407; A61K 9/7053; A61K 47/06

(57) ABSTRACT

A method for reducing skin sensitization of an asenapine-containing patch comprising a backing layer and an adhesive agent layer, the method comprising:
using an adhesive agent for preparing the adhesive agent layer, the adhesive agent containing free asenapine in a range of 2 to 5% by mass, styrene-isoprene-styrene block copolymer (SIS) in a range of 7 to 18% by mass, polyisobutylene (PIB) in a range of 0.5 to 10% by mass, alicyclic saturated hydrocarbon resin in a range of 30 to 70% by mass, and liquid paraffin in a range of 5 to 10% by mass, relative to a total amount of the adhesive agent; and
preparing the asenapine-containing patch whose average cumulative amount of skin permeation of asenapine (24 hours) after applying the asenapine-containing patch to skin of a dorsal scapular region of a 5-week-old Hartley white female guinea pig and holding the asenapine-containing patch with an occlusive dressing for 24 hours is in a range of 90 to 300 μg/cm² in terms of free form.

### BACKGROUND OF THE INVENTION

### Field of the Invention

The present invention relates to a method for reducing skin sensitization of an asenapine-containing patch comprising a backing layer and an adhesive agent layer.

### Related Background Art

Asenapine (trans-5-chloro-2-methyl-2,3,3a,12b-tetrahydro-1H-dibenzo[2,3:6,7]oxepino[4,5-c]pyrrole) is a compound having a central nervous system (CNS) suppressing activity, an anti-histamine activity, and an anti-serotonin activity and is known as a drug used in the treatment of central nervous system diseases such as schizophrenia.

As asenapine-containing pharmaceutical formulations, for example, WO2010/127674A1 describes topical formulations such as sprays, aerosols, patches, and ointments. In addition, US2015/0202183A1, US2015/0231250A1, US2015/0164862A1, US2017/0035732A1, US2017/0172981A1, US2018/0289631A1, US2018/0339050A1, US2018/0360968A1, US2019/0105391A1, and US2019/0343800A1 describe patches containing asenapine.

### SUMMARY OF THE INVENTION

The present inventors found a problem that even when the content of asenapine in a patch containing asenapine as a drug and the composition of its adhesive agent layer are similar, there are cases where sensitization caused by the drug against the skin is observed and cases where no such sensitization is observed.

The present invention has been made in view of the above-described problem and an object of the present invention is to provide a method, for a patch containing asenapine as a drug, for sufficiently reducing sensitization caused by the drug against the skin.

The present inventors found that in an asenapine-containing patch comprising: an adhesive agent layer that is obtained using an adhesive agent having a specific composition; and a backing layer, not only the content of asenapine in the adhesive agent layer laminated on the backing layer and the composition of the adhesive agent layer but also the cumulative amount of skin permeation of asenapine into the skin of a guinea pig have a relation with sensitization caused by the drug against the skin. The present inventors then found that it was possible to achieve the above-described object by setting the average cumulative amount of skin permeation of asenapine in the asenapine-containing patch (24 hours in terms of free form) into the skin of a guinea pig within a specific range, and finally completed the present invention.

The present invention is a method for reducing skin sensitization of an asenapine-containing patch comprising a backing layer and an adhesive agent layer, the method comprising:

using an adhesive agent for preparing the adhesive agent layer, the adhesive agent containing free asenapine in a range of 2 to 5% by mass, styrene-isoprene-styrene block copolymer (SIS) in a range of 7 to 18% by mass, polyisobutylene (PIB) in a range of 0.5 to 10% by mass, alicyclic saturated hydrocarbon resin in a range of 30 to 70% by mass, and liquid paraffin in a range of 5 to 10% by mass, relative to a total amount of the adhesive agent; and

preparing the asenapine-containing patch whose average cumulative amount of skin permeation of asenapine (24 hours) after applying the asenapine-containing patch to skin of a dorsal scapular region of a 5-week-old Hartley white female guinea pig and holding the asenapine-containing patch with an occlusive dressing for 24 hours is in a range of 90 to 300 μg/cm² in terms of free form.

In the present invention, it is preferred that an amount of the adhesive agent layer after drying is in a range of 80 to 400 g/m².

Further, in the present invention, it is preferred that the adhesive agent further contains isopropyl palmitate in a range of 1 to 20% by mass.

According to the present invention, it is possible, for a patch containing asenapine as a drug, to sufficiently reduce sensitization caused by the drug against the skin.

Note that in the present invention, the average cumulative amount of skin permeation of asenapine (24 hours in terms of free form) is a value derived by the following method.

(Measurement of Average Cumulative Amount of Skin Permeation of Asenapine)

For an asenapine-containing patch (size: 2 cm×2 cm) as a test patch, the average cumulative amount of skin permeation of asenapine (24 hours in terms of free form) was measured as below using 5-week-old Hartley white female guinea pigs (weight: 326 to 402 g, 6 guinea pigs) under a test environment of a temperature of 19 to 25° C. and a relative humidity of 30 to 70%.

First, on Day 0 of the measurement, the dorsal scapular region of each guinea pig was cleared of hair, and the test patch was applied onto the skin of this region and held by an occlusive dressing for 24 hours, and then the test patch was peeled off from the skin. Subsequently, the same treatment as that applied on Day 0 of the measurement was applied to the same dorsal scapular region of the guinea pig on Day 7 of the measurement. Moreover, the same treatment as that applied on Day 0 of the measurement was applied to the same dorsal scapular region of the guinea pig on Day 14 of the measurement as well.

Thereafter, the cumulative amount of permeation of asenapine (in terms of free form) that permeated the skin of the guinea pig during the 24 hours of application of the test patch to its skin, i.e., the cumulative amount of skin permeation of asenapine (24 hours in terms of free form), was measured as below.

Patches (unused patches) cut out into a size of 2 cm×2 cm (4 cm²) and the patches (peeled-off patches) peeled off from the guinea pigs were each put into an extraction container, into which 10 mL of tetrahydrofuran (THF) was added, followed by shaking for 60 minutes to extract asenapine (free form) into the THF. Then, a diluent (methanol: 0.01 mol/L SDS (1000-times diluted phosphoric acid)=3:1) was added to adjust the solution in the container precisely to 50 mL. Then, the solution thus obtained was filtrated using a membrane filter to obtain a test liquid. Each test liquid thus obtained was analyzed under the following conditions using a high-performance liquid chromatography (HPLC) to quantify the content of asenapine in each patch (in terms of free form). Then, the cumulative amount of skin permeation of asenapine (24 hours in terms of free form) was derived based on the following formula (1).

The cumulative amount of skin permeation of asenapine [μg/cm²]={(the content of asenapine in the unused patch [μg])−(the content of asenapine in the peeled-off patch [μg])}/4 [cm²] (1)

Then, the average value of the cumulative amounts of skin permeation of asenapine (24 hours in terms of free form) on Days 0, 7, and 14 of the measurement derived for each guinea pig used in the test was derived. Moreover, the average value of the cumulative amounts of skin permeation of asenapine (24 hours in terms of free form) derived for all guinea pigs used in the test was determined as the average cumulative amount of skin permeation of asenapine (24 hours in terms of free form).

<HPLC Conditions>

### Column: TSKgelODS-80TsQA 5 μm (4.6 mm I.D.×150 mm)

Mobile phase: mixture liquid (methanol: 0.01 mol/L SDS (1000-times diluted phosphoric acid)=3:1)
Measurement wavelength: 230 nm
Column temperature: 40° C.
Flow rate: 1.0 mL/min
Introduced amount: 10 μL

### DETAILED DESCRIPTION OF THE PREFERRED EMBODIMENTS

Hereinafter, the present invention is described in further detail according to the preferred embodiments.

The present invention is a method for reducing skin sensitization of an asenapine-containing patch comprising a backing layer and an adhesive agent layer, the method comprising:

using an adhesive agent for preparing the adhesive agent layer, the adhesive agent containing free asenapine in a range of 2 to 5% by mass, styrene-isoprene-styrene block copolymer (SIS) in a range of 7 to 18% by mass, polyisobutylene (PIB) in a range of 0.5 to 10% by mass, alicyclic saturated hydrocarbon resin in a range of 30 to 70% by mass, and liquid paraffin in a range of 5 to 10% by mass, relative to a total amount of the adhesive agent; and

preparing the asenapine-containing patch whose average cumulative amount of skin permeation of asenapine (24 hours) after applying the asenapine-containing patch to skin of a dorsal scapular region of a 5-week-old Hartley white female guinea pig and holding the asenapine-containing patch with an occlusive dressing for 24 hours is in a range of 90 to 300 μg/cm² in terms of free form.

The asenapine-containing patch according to the present invention comprises a backing layer and an adhesive agent layer disposed on at least one surface of the backing layer. As the backing layer, a conventionally-known one may be used as appropriate, and the material of such a backing layer includes, for example, synthetic resins such as polyethylene, polypropylene, polybutadiene, ethylene-vinyl acetate copolymer, vinyl acetate-vinyl chloride copolymer, polyvinyl chloride, polyamides (such as nylon), polyester, cellulose derivatives, and polyurethane. In addition, the form of the backing layer includes a film; a sheet; a sheet-shaped porous body; a sheet-shaped foam; fabrics such as a woven fabric, a braided fabric, and a nonwoven fabric; stacked bodies of these; and the like. In the present invention, the thickness of the backing layer is not particularly limited, but it is usually preferable that the thickness be approximately in a range of 2 to 3000 μm.

Moreover, a release liner may be further included on a surface of the adhesive agent layer on the opposite side to the backing layer. Such a release liner may be any release liner as long as the release liner covers the adhesive agent layer until the patch is used and can be peeled off and removed when the patch is used, and may be paper (release paper) or an aluminum foil. Alternatively, its material may be a synthetic resin. Specifically, the release liner includes polyesters such as polyethylene terephthalate and polyethylene naphthalate; polyolefins such as polyethylene and polypropylene; films of polyvinyl chloride, polyvinylidene chloride, and the like; laminate films of wood-free papers and polyolefins; films of nylon, aluminum, and the like; and the like. As these release liners, it is preferable to use those which are subjected to surface coating with a release agent of silicone, polytetrafluoroethylene, or the like (releasing treatment) from the viewpoint of facilitating releasing from the adhesive agent layer.

The adhesive agent for preparing the adhesive agent layer of the asenapine-containing patch according to the present invention comprises free asenapine in a range of 2 to 5% by mass, styrene-isoprene-styrene block copolymer in a range of 7 to 18% by mass, polyisobutylene in a range of 0.5 to 10% by mass, alicyclic saturated hydrocarbon resin in a range of 30 to 70% by mass, and liquid paraffin in a range of 5 to 10% by mass, relative to a total amount of the adhesive agent.

<Asenapine>

The asenapine according to the present invention refers to trans-5-chloro-2-methyl-2,3,3a,12b-tetrahydro-1H-dibenzo[2,3:6,7]oxepino[4,5-c]pyrrole. Asenapine has a central nervous system (CNS) suppressing activity, an anti-histamine activity, and an anti-serotonin activity and is generally known as a drug used in the treatment of central nervous system diseases such as schizophrenia.

In the present invention, such asenapine is required to be free asenapine which is in the free form. When free asenapine is used as a drug, combination with styrene-isoprene-styrene block copolymer and polyisobutylene, which are rubber-based adhesive agents described later improves the skin permeability of the drug.

The free asenapine according to the present invention may be free asenapine added at the time of production of a patch or may be generated from a pharmaceutically acceptable salt of asenapine in the adhesive agent layer from the viewpoint of the handleability and stability of the raw material. Alternatively, the free asenapine may be a mixture of these. The method for generating the free asenapine from a pharmaceutically acceptable salt of asenapine (hereinafter sometimes referred to as an asenapine salt) includes, for example, a method including blending the asenapine salt and a metal ion-containing desalting agent (neutralizing agent) in the composition of the adhesive agent layer at the time of production of a patch to desalinate the asenapine salt.

As the asenapine salt, an acid adduct is preferable from the viewpoint that the acid adduct is easily desalinated by the metal ion-containing desalting agent. The acid includes monobasic acids such as hydrochloric acid, hydrobromic acid, and methanesulfonic acid; and polybasic acids such as fumaric acid, maleic acid, citric acid, and tartaric acid. One of these may be used alone or two or more of these may be used in combination.

In addition, the metal ion-containing desalting agent includes metal hydroxides, acetic acid metal salts, and the like. The metal includes sodium, potassium, magnesium, and the like. One of these may be used alone or two or more of these may be used in combination. Among these, the metal ion-containing desalting agent is preferably an alkali metal ion-containing desalting agent and particularly preferably sodium hydroxide and sodium acetate, from the viewpoint that they are easy to handle at the production and further improve the stability over time of the free asenapine.

In a case where the free asenapine according to the present invention is generated from the asenapine salt, the amount of the metal ion-containing desalting agent blended is preferably in a range of 0.5 to 6 equivalents and more preferably in a range of 1 to 6 equivalents relative to the acid-base equivalent of the asenapine salt. If the amount of the metal ion-containing desalting agent blended is less than the lower limit, it becomes difficult to generate the free asenapine in a sufficient amount, making it difficult to achieve a sufficient skin permeability. On the other hand, if the amount is more than the upper limit, the adhesive force of the adhesive agent layer tends to decrease.

In the adhesive agent according to the present invention, the content of the free asenapine needs to be in a range of 2 to 5% by mass relative to the total amount of the adhesive agent. If the content of the free asenapine is less than the lower limit, the skin permeation amount becomes insufficient, so that the area of the patch needs to be increased. On the other hand, if the content is more than the upper limit, local side effects such as skin irritation tend to occur or skin sensitization becomes likely to be observed. Moreover, from the same viewpoint, the content of the free asenapine is particularly preferably in a range of 3 to 4% by mass relative to the total amount of the adhesive agent.

Further, in the adhesive agent according to the present invention, the content of the free asenapine per unit area (g/m²) is preferably in a range of 1.6 to 20 g/m² and more preferably in a range of 2.0 to 10 g/m². If the content of the free asenapine per unit area is less than the lower limit, the amount of skin permeation is insufficient, so that the patch tends to require a larger area. On the other hand, if the content is more than the upper limit, local side effects such as skin irritation tend to occur or skin sensitization tends to be observed.

<Rubber-Based Adhesive Agent>

In the adhesive agent according to the present invention, as the rubber-based adhesive agent, which is an adhesive base agent, it is necessary to use styrene-isoprene-styrene block copolymer (SIS) and polyisobutylene (PIB) in combination. Using the SIS and the PIB in combination with the free asenapine in this way makes it possible to achieve both an improvement in the releasability of asenapine from the adhesive agent layer and an improvement in the adhesive force of the adhesive agent layer.

In the adhesive agent according to the present invention, the content of the SIS needs to be in a range of 7 to 18% by mass relative to the total amount of the adhesive agent. If the content of the SIS is less than the lower limit, the adherability of the patch to the skin decreases. On the other hand, the content is more than the upper limit, the releasability of asenapine from the adhesive agent layer decreases, making it difficult to achieve a sufficient skin permeability. Moreover, from the same viewpoint, the content of the SIS is particularly preferably in a range of 9 to 16% by mass relative to the total amount of the adhesive agent.

In the adhesive agent according to the present invention, the content of the PIB needs to be in a range of 0.5 to 10% by mass relative to the total amount of the adhesive agent. If the content of the PIB is less than the lower limit, the adherability of the patch to the skin decreases. On the other hand, the content is more than the upper limit, the releasability of asenapine from the adhesive agent layer decreases, making it difficult to achieve a sufficient skin permeability. Moreover, from the same viewpoint, the content of the PIB is particularly preferably in a range of 3 to 8% by mass relative to the total amount of the adhesive agent.

In addition, a mass ratio between the SIS and the PIB (SIS:PIB) is preferably in a range of 4:1 to 3:2 and more preferably in a range of 3:1 to 2:1 from the viewpoint of achieving both an improvement in the releasability of asenapine from the adhesive agent layer and an improvement in the adhesive force of the adhesive agent layer. Moreover, a mass ratio between the free asenapine and the rubber-based adhesive agent (the total amount of the SIS and the PIB) (free asenapine:rubber-based adhesive agent) is preferably in a range of 1:3 to 1:14 and more preferably in a range of 1:4 to 1:8 from the viewpoint of achieving both an improvement in the releasability of asenapine from the adhesive agent layer and an improvement in the adhesive force of the adhesive agent layer.

<Tackifier>

In the adhesive agent according to the present invention, it is necessary to use alicyclic saturated hydrocarbon resin as a tackifier. Using the alicyclic saturated hydrocarbon resin in combination with the free asenapine, the SIS, and the PIB in this manner makes it possible to improve the adherability of the patch to the skin.

In the adhesive agent according to the present invention, the content of the alicyclic saturated hydrocarbon resin needs to be in a range of 30 to 70% by mass relative to the total amount of the adhesive agent. If the content of the alicyclic saturated hydrocarbon resin is less than the lower limit, the adherability of the patch to the skin decreases. On the other hand, if the content is more than the upper limit, the cohesive force of the adhesive agent layer decreases, making it likely that the pain during the peeling-off increases. In addition, from the same viewpoint, the content of the alicyclic saturated hydrocarbon resin is particularly preferably in a range of 45 to 65% by mass relative to the total amount of the adhesive agent.

In addition, the mass ratio between the rubber-based adhesive agent (the total amount of the SIS and the PIB) and the alicyclic saturated hydrocarbon resin (rubber-based adhesive agent:alicyclic saturated hydrocarbon resin) is preferably in a range of 1:1.1 to 1:5 and more preferably in a range of 1:2 to 1:4 from the viewpoint of achieving both suppression of decrease in the attachment force to the skin and suppression of decrease in the cohesive force of the adhesive agent layer. Moreover, the mass ratio between the free asenapine and the alicyclic saturated hydrocarbon resin (free asenapine:alicyclic saturated hydrocarbon resin) is preferably in a range of 1:6 to 1:30 and more preferably in a range of 1:8 to 1:25 from the viewpoint of suppressing decrease in the attachment force to the skin.

<Softener>

In the adhesive agent according to the present invention, it is necessary to use liquid paraffin as a softener. Using the liquid paraffin in combination with the free asenapine, the SIS, the PIB, and the alicyclic saturated hydrocarbon resin in this manner makes it possible to improve the adherability of the patch to the skin and the pharmaceutical physical properties thereof.

In the adhesive agent according to the present invention, the content of the liquid paraffin needs to be in a range of 5 to 10% by mass relative to the total amount of the adhesive agent. If the content of the liquid paraffin is less than the lower limit, the adherability of the patch to the skin decreases. On the other hand, if the content is more than the upper limit, the cohesive force of the adhesive agent layer decreases, so that the adhesive agent layer or stickiness tends to remain on the skin after the peeling-off. In addition, from the same viewpoint, the content of the liquid paraffin particularly preferably in a range of 6 to 9% by mass relative to the total amount of the adhesive agent.

In addition, the mass ratio between the rubber-based adhesive agent (the total amount of the SIS and the PIB) and the liquid paraffin (rubber-based adhesive agent:liquid paraffin) is preferably in a range of 4:1 to 1:1 and more preferably in a range of 3:1 to 2:1 from the viewpoint of achieving both suppression of decrease in the attachment force to the skin and suppression of decrease in the cohesive force of the adhesive agent layer. Moreover, the mass ratio between the free asenapine and the liquid paraffin (free asenapine:liquid paraffin) is preferably in a range of 1:1 to 1:5 and more preferably in a range of 1:2 to 1:4 from the viewpoint of suppressing decrease in the attachment force to the skin.

<Absorption Enhancer>

Preferably, the adhesive agent according to the present invention further contain an absorption enhancer. The absorption enhancer may be a compound conventionally known to have a transdermal absorption enhancing effect. Examples of the absorption enhancer include organic acid esters (e.g., fatty acid esters and cinnamic acid esters), organic acid amides (e.g., fatty acid amides), aliphatic alcohols, polyalcohols, ethers (e.g., aliphatic ethers and polyoxyethylene alkyl ethers), and the like. These absorption enhancers may have an unsaturated bond, and may have a cyclic, linear, or branched chemical structure. Also, the absorption enhancer may be a monoterpene-based compound, a sesquiterpene-based compound, or a vegetable oil (e.g., olive oil). One of these absorption enhancers may be used alone, or two or more may be used in combination.

More preferably, the adhesive agent according to the present invention further contains isopropyl palmitate as an absorption enhancer. Using the isopropyl palmitate in combination with the free asenapine, the SIS, and the PIB in this manner makes it possible to improve the skin permeability of asenapine.

In the adhesive agent according to the present invention, the content of the isopropyl palmitate is preferably in a range of 1 to 20% by mass relative to the total amount of the adhesive agent. If the content of the isopropyl palmitate is less than the lower limit, the releasability of asenapine from the adhesive agent layer tends to decreases. On the other hand, if the content of the isopropyl palmitate is more than the upper limit, the absorption enhancer tends to be separated from the adhesive agent layer and impair the adhesiveness of the adhesive agent layer and local side effects such as skin irritation tend occur. In addition, from the same viewpoint, the content of the isopropyl palmitate is particularly preferably in a range of 5 to 12% by mass relative to the total amount of the adhesive agent.

In addition, the mass ratio between the free asenapine and the isopropyl palmitate (free asenapine:isopropyl palmitate) is preferably in a range of 1:1 to 1:5 and more preferably in a range of 1:2 to 1:4 from the viewpoint of achieving both an improvement in the releasability of asenapine from the adhesive agent layer and a reduction of skin sensitization.

<Other Additive and the Like>

As described above, the adhesive agent for preparing the adhesive agent layer of the asenapine-containing patch according to the present invention comprises the free asenapine, the styrene-isoprene-styrene block copolymer, the polyisobutylene, the alicyclic saturated hydrocarbon resin, and the liquid paraffin (and the isopropyl palmitate as necessary) in the respective predetermined ranges, but may further comprise an additive such as a stabilizer as necessary within a range that does not hinder the effect of the present invention.

Such a stabilizer includes tocopherol and ester derivatives thereof, ascorbic acid and ester derivatives thereof, dibutylhydroxytoluene, butylated hydroxyanisole, and the like. One of these may be used alone or two or more of these may be used in combination. Among these, it is more preferable to use dibutylhydroxytoluene as a stabilizer from the viewpoint of pharmaceutical physical properties and external appearance, and drug stabilizing effect. When the adhesive agent according to the present invention comprises such a stabilizer, the content of the stabilizer is preferably in a range of 0.1 to 3% by mass relative to the total amount of the adhesive agent. If the content of the stabilizer is less than the lower limit, the stability of each component in the patch tends to decrease. On the other hand, if the content is more than the upper limit, the cohesive force of the adhesive agent layer tends to decrease.

In addition, the adhesive agent for preparing the adhesive agent layer of the asenapine-containing patch according to the present invention preferably contains substantially no water. Since the adhesive agent according to the present invention mainly composed of hydrophobic components, if the content of water is more than 10% by mass, the water content tends to be separated from the adhesive agent layer to impair the adhesiveness of the adhesive agent layer. Here, containing substantially no water means that no water is intentionally added at the time of production, and the content of water measured by the Karl Fischer Method according to the Japanese Pharmacopoeia is less than 10% by mass relative to the total amount of the adhesive agent layer.

<Method for Reducing Skin Sensitization of Asenapine-Containing Patch>

In the present invention, an adhesive agent layer made of the adhesive agent is prepared, and moreover the asenapine-containing patch is prepared whose average cumulative amount of skin permeation of asenapine (24 hours) after applying the asenapine-containing patch to the skin of the dorsal scapular region of a 5-week-old Hartley white female guinea pig and holding the asenapine-containing patch with an occlusive dressing for 24 hours is in a range of 90 to 300 μg/cm² in terms of free form. In this way, the skin sensitization caused by the drug in the case of applying the asenapine-containing patch thus obtained to the skin is sufficiently reduced. In other words, if the average cumulative amount of skin permeation of asenapine is less than the lower limit, the amount of the drug transferred at the time of application decreases. On the other hand, if the amount of skin permeation of asenapine is more than the upper limit, reduction of the sensitization caused by the drug against the skin becomes unlikely to be sufficiently achieved. Also, from the same viewpoint, the average cumulative amount of skin permeation of asenapine is particularly preferably in a range of 150 to 300 μg/cm² in terms of free form.

In the present invention, the average cumulative amount of skin permeation of asenapine (24 hours in terms of free form) is a value derived by the above-described method. By deriving the average cumulative amount of skin permeation of asenapine (24 hours in terms of free form) fora test patch prepared in advance by the above-described method, and thereafter preparing a patch with the same specifications as the test patch, it is possible to prepare the asenapine-containing patch according to the present invention which satisfies the above-described condition (the average cumulative amount of skin permeation of asenapine (24 hours in terms of free form) is in a range of 90 to 300 μg/cm²).

<Method for Producing Asenapine-Containing Patch>

A specific method for producing the asenapine-containing patch according to the present invention is not particularly limited, and the asenapine-containing patch can be produced by employing a publicly known method for producing a patch as appropriate.

For example, first, the free asenapine, the rubber-based adhesive agent (the SIS and the PIB), the alicyclic saturated hydrocarbon resin, and the liquid paraffin (and the isopropyl palmitate as necessary) are kneaded together with a solvent in accordance with a conventional method to obtain a uniform adhesive agent layer composition. Then, this adhesive agent layer composition is applied onto a surface (usually, onto one surface) of the backing layer to a predetermined thickness, followed as necessary by heating to dry and remove the solvent, and the resultant is cut into a desired size, so that the asenapine-containing patch can be obtained. Note that the solvent used to obtain the adhesive agent layer composition includes, for example, toluene, ethanol, methanol, ethyl acetate, and the like. One of these may be used alone or two or more of these may be used in combination. In addition, the conditions for the heating may be selected as appropriate depending on the solvent; however, the temperature condition is preferably 60 to 120° C. in general and the heating time is preferably 2 to 30 minutes in general.

In addition, when the asenapine-containing patch is prepared, the method may further comprise a step of laminating the release liner on a surface of the adhesive agent layer on the opposite side to the backing layer. Moreover, a step of first applying the adhesive agent layer composition onto one surface of the release liner to the predetermined thickness to form the adhesive agent layer and thereafter laminating the backing layer on the surface of the adhesive agent layer on the opposite side to the release liner may be employed.

<Amount of Adhesive Agent Layer>

In the present invention, when the adhesive agent layer composition is applied onto a surface of the backing layer, it is preferred to make the amount of the adhesive agent layer after drying in a range of 80 to 400 g/m². In the asenapine-containing patch comprising the adhesive agent layer obtained by using the adhesive agent having the specific composition, and the backing layer as described above, making the amount of the adhesive agent layer laminated on the backing layer in the above-described range makes it possible to more sufficiently reduce the sensitization caused by the drug against the skin. In other words, if the amount of the adhesive agent layer is less than the lower limit, the amount of the drug transferred at the time of application tends to decrease. On the other hand, if the amount of the adhesive agent layer is more than the upper limit, reduction of the sensitization caused by the drug against the skin tends not to be sufficiently achieved. In addition, from the same viewpoint, the amount of the adhesive agent layer after drying is particularly preferably in a range of 90 to 300 g/m².

<Area of Patch>

In the present invention, the area of the asenapine-containing patch is preferably in a range of 5 to 160 cm² and more preferably in a range of 10 to 80 cm². If the area of the patch is less than the lower limit, it tends to be difficult to achieve a sufficient skin permeability. On the other hand, if the area of the asenapine-containing patch is more than the upper limit, the attachment force to the skin during application tends to decrease.

<Skin Sensitization and Method for Evaluating Skin Sensitization>

Skin sensitization is one of delayed-type hypersensitivities and is a phenomenon that causes rash on the skin through an excessive immune reaction due to a chemical substance. Even with a slight contact that does not exhibit primary irritation, the sensitized T-cells in the lymph nodes proliferate due to a matter having the sensitization potential, and when the skin touches the same chemical substance again, the recognized T-cells release lymphokine to cause skin inflammation, so that the skin sensitization becomes positive.

As methods for testing such skin sensitization, the Maximisation Test (Guinea Pig Maximisation Test (GPMT)), the Buehler Test, and the Local Lymph Node Assay (LLNA) are known, and any of these methods can be used for evaluating skin sensitization in the present invention. However, the Buehler Test is preferably employed from the viewpoint that this method allows evaluation only with the application to the skin. Note that the Maximisation Test and the Buehler Test are listed as TG406 of the “Organization for Economic Co-operation and Development (OECD) GUIDELINE FOR TESTING OF CHEMICALS” and the Local Lymph Node Assay is listed as TG429 of the same GUIDELINE.

### EXAMPLES

Although the present invention is described below in more detail based on Examples and Comparative Examples, the present invention is not limited to Examples described below. Note that for an asenapine-containing patch obtained in each of Examples and Comparative Examples, the average cumulative amount of skin permeation of asenapine (24 hours in terms of free form) was derived by the above-described method, and moreover skin sensitization was evaluated by the following method in accordance with the Buehler Test listed as TG406 of “Organization for Economic Co-operation and Development (OECD) GUIDELINE FOR TESTING OF CHEMICALS”.

(Method for Evaluating Skin Sensitization)

For the asenapine-containing patch (test patch, size: 2 cm×2 cm) obtained in each of Examples and Comparative Examples, skin sensitization was evaluated as described below in accordance with the Buehler Test using 5-week-old Hartley white female guinea pigs (weight: 326 to 402 g, sensitization group: 6 guinea pigs, control group (non-sensitization group): 6 guinea pigs).

<Test Environment and Conditions>

### Temperature: 19 to 25° C.

Relative humidity: 30 to 70%

<Sensitization>

The sensitization of the guinea pigs of the sensitization group was conducted as follows. Specifically, the dorsal scapular regions of the guinea pigs were cleared of hair on Day 0 of the sensitization, and the test patches were applied onto the skins of these regions and held by an occlusive dressing for 24 hours. On the other hand, as the control group (non-sensitization group), guinea pigs that were not subjected to the above-described treatment were used as the untreated control group. Subsequently, the same treatment as that applied on Day 0 of the sensitization was applied to the same dorsal scapular regions of the guinea pigs of the sensitization group on Day 7 of the sensitization. Moreover, the same treatment as that applied on Day 0 of the sensitization was applied to the same dorsal scapular regions of the guinea pigs of the sensitization group on Day 14 of the sensitization as well.

<Challenge>

The challenge was performed by clearing the hair of flanks of the guinea pigs of the sensitization group and the control group (non-sensitization group) on Day 28 of the sensitization and applying the test patches onto the skins of the regions and holding them with an occlusive dressing for 24 hours.

<Evaluation>

The challenge areas were cleared of hair 24 hours after and 48 hours after removing the test patches and the skin reaction was observed. The results of the observation were judged based on the skin reaction evaluation criterion of Draize shown in Table 1 to obtain the skin reaction scores (average values) 24 hours after removing the patch and 48 hours after removing the patch, respectively.

TABLE 1
Draize Skin Reaction Evaluation Criteria

| | | Evaluation |
|---|---|---|
| Observed Item | Degree | Value (*1) |
| Erythema and | No erythema | 0 |
| eschar | Very slight erythema (barely perceptible) | 1 |
| formation | Well-defined erythema | 2 |
| | Moderate-to-severe erythema | 3 |
| | Severe erythema (beet redness) to slight | 4 |
| | eschar formation (injuries in depth) | |
| Edema formation | No edema | 0 |
| | Very slight edema (barely perceptible) | 1 |
| | Slight edema (edges of area well defined | 2 |
| | by definite raising) | |
| | Moderate edema (raised about 1 mm) | 3 |
| | Severe edema (raised more than 1 mm and | 4 |
| | extending beyond area of exposure) | |

*1: Skin reaction score = The total point of the evaluation point of “erythema and eschar formation” and the evaluation point of “edema formation”

### Example 1

First, 14.1 parts by mass of styrene-isoprene-styrene block copolymer (SIS), 6.0 parts by mass of polyisobutylene (PIB), 58.6 parts by mass of alicyclic saturated hydrocarbon resin (trade name: Arkon, manufactured by Arakawa Chemical Industries, Ltd.), and 8.1 parts by mass of liquid paraffin were mixed to obtain a uniform adhesive base agent composition. Next, 3.2 parts by mass of free asenapine, 10.0 parts by mass of isopropyl palmitate (IPP), 86.8 parts by mass of the above adhesive base agent composition, and an appropriate amount of toluene were mixed to obtain a uniform adhesive agent layer composition. The composition of the adhesive agent layer composition thus obtained (exclusive of the toluene) is as shown in Table 2.

Next, this adhesive agent layer composition was applied onto one surface of a polyester film (release liner) subjected to a peeling process and having a thickness of 75 μm such that the amount of the adhesive agent layer composition after drying became 100 g/m², followed by drying at 80° C. for 15 minutes to remove the toluene and form an adhesive agent layer. Next, a PET film (backing layer) having a thickness of 25 μm was laminated on a surface of the adhesive agent layer on the opposite side to the release liner, followed by cutting to obtain an asenapine-containing patch.

### Example 2 and Comparative Examples 1 to 5

Asenapine-containing patches were obtained in the same manner as in Example 1 except that the compositions of their adhesive agent layer compositions were the compositions shown in Table 2 and also that the amounts of their adhesive agent layers after drying were the amounts shown in Table 2.

### Comparative Example 6

First, 3.5 parts by mass of free asenapine, 96.5 parts by mass of a first acrylic-based adhesive agent (trade name: DURO-TAK 87-4287 (DT-4287), manufactured by Henkel AG & Co. KGaA), and an appropriate amount of toluene were mixed to obtain a uniform adhesive agent layer composition. The composition of the adhesive agent layer composition thus obtained (exclusive of the toluene) is as shown in Table 3.

Next, this adhesive agent layer composition was applied onto one surface of a polyester film (release liner) subjected to a peeling process and having a thickness of 75 μm such that the amount of the adhesive agent layer composition after drying became 100 g/m², followed by drying at 80° C. for 15 minutes to remove the toluene and form an adhesive agent layer. Next, a PET film (backing layer) having a thickness of 25 μm was laminated on a surface of the adhesive agent layer on the opposite side to the release liner, followed by cutting to obtain an asenapine-containing patch.

### Comparative Examples 7 to 10

Asenapine-containing patches were obtained in the same manner as in Comparative Example 6 except that the compositions of their adhesive agent layer compositions were the compositions shown in Table 3 and also that the amounts of their adhesive agent layers after drying were the amounts shown in Table 3. Note that a second acrylic-based adhesive agent with a trade name DURO-TAK 87-4098 (DT-4098), manufactured by Henkel AG & Co. KGaA, and a silicone-based adhesive agent with a trade name BIO-PSA 7-4202 (BIO-PSA 4202), manufactured by DuPont Toray Specialty Materials K.K., were used.

(Evaluation)

For the asenapine-containing patch obtained in each of Examples and Comparative Examples, the average cumulative amount of skin permeation of asenapine (24 hours in terms of free form) was derived by the above-described method. Moreover, the skin sensitization was evaluated by the above-described method. The results thus obtained are shown in Tables 2 and 3.

TABLE 2

| | | | Comparative | Comparative | | Comparative | Comparative | Comparative |
|---|---|---|---|---|---|---|---|---|
| | | Example 1 | Example 1 | Example 2 | Example 2 | Example 3 | Example 4 | Example 5 |
| Composition | Free asenapine | 3.2 | 6.5 | 10.0 | 3.2 | 3.5 | 10.0 | 4.0 |
| [% by mass] | Styrene-isoprene-styrene | 14.1 | 15.2 | 14.7 | 14.1 | 96.5 | 90.0 | — |
| | block copolymer (SIS) | | | | | | | |
| | Polyisobutylene (PIB) | 6.0 | 6.5 | 6.3 | 6.0 | — | — | 96.0 |
| | Alicyclic saturated | 58.6 | 63.1 | 60.7 | 58.6 | — | — | — |
| | hydrocarbon resin | | | | | | | |
| | Liquid paraffin | 8.1 | 8.7 | 8.4 | 8.1 | — | — | — |
| | Isopropyl palmitate | 10.0 | — | — | 10.0 | — | — | — |
| | Total | 100 | 100 | 100 | 100 | 100 | 100 | 100 |
| The mass of the adhesive agent layer | | 100 | 100 | 100 | 200 | 100 | 100 | 100 |
| [g/m²] | | | | | | | | |
| Average cumulative amount of skin | | 185.2 | 321.9 | 462.7 | 286.9 | 179.4 | 659.8 | 245.8 |
| permeation of asenapine (24 hours in | | | | | | | | |
| terms of free form) [μg/cm²] | | | | | | | | |
| Skin | The skin reaction score | 0.0/0.3 | 0.0/0.0 | 0.0/0.0 | 0.0/— | 0.2/0.0 | 0.0/0.0 | 0.2/0.0 |
| sensitization | (sensitization | | | | | | | |
| test | group/non-sensitization | | | | | | | |
| | group, 24 hours after | | | | | | | |
| | removing the patch) | | | | | | | |
| | The skin reaction score | 0.0/0.0 | 0.2/0.0 | 0.3/0.0 | 0.0/— | 0.7/0.0 | 0.3/0.0 | 0.2/0.0 |
| | (sensitization | | | | | | | |
| | group/non-sensitization | | | | | | | |
| | group, 48 hours after | | | | | | | |
| | removing the patch) | | | | | | | |
| | Skin sensitization | negative | positive | positive | negative | positive | positive | positive |

TABLE 3

| | | Comparative | Comparative | Comparative | Comparative | Comparative |
|---|---|---|---|---|---|---|
| | | Example 6 | Example 7 | Example 8 | Example 9 | Example 10 |
| Composition | Free asenapine | 3.5 | 5.0 | 3.5 | 4.0 | 10.0 |
| [% by mass] | Acrylic-based adhesive | 96.5 | 95.0 | 96.5 | — | — |
| | agent (DT-4287) | | | | | |
| | Acrylic-based adhesive | — | — | — | 96.0 | — |
| | agent (DT-4098) | | | | | |
| | Silicone-based adhesive | — | — | — | — | 90.0 |
| | agent (BIO-PSA 4202) | | | | | |
| | Total | 100 | 100 | 100 | 100 | 100 |
| The mass of the adhesive agent layer | | 100 | 100 | 200 | 100 | 100 |
| [g/m²] | | | | | | |
| Average cumulative amount of skin | | 184.6 | 293.5 | 154.4 | 215.2 | 796.1 |
| permeation of asenapine (24 hours in | | | | | | |
| terms of free form) [μg/cm²] | | | | | | |
| Skin | The skin reaction score | 0.3/0.0 | 0.3/0.0 | 0.0/0.0 | 0.2/0.0 | 0.7/0.7 |
| sensitization | (sensitization | | | | | |
| test | group/non-sensitization | | | | | |
| | group, 24 hours after | | | | | |
| | removing the patch) | | | | | |
| | The skin reaction score | 0.7/0.0 | 0.7/0.0 | 0.5/0.0 | 0.5/0.0 | 0.7/0.3 |
| | (sensitization | | | | | |
| | group/non-sensitization | | | | | |
| | group, 48 hours after | | | | | |
| | removing the patch) | | | | | |
| | Skin sensitization | positive | positive | positive | positive | positive |

As is clear from the results shown in Tables 2 and 3, in the asenapine-containing patch comprising: the adhesive agent layer obtained using the adhesive agent having the specific composition in the range of the present invention; and the backing layer, in the cases where the average cumulative amount of skin permeation of asenapine (24 hours in terms of free form) was set in the range of the present invention (Examples 1 and 2), sensitization caused by the drug against the skin was obviously reduced as compared with the cases where the average cumulative amount of skin permeation of asenapine (24 hours in terms of free form) was out of the range of the present invention (Comparative Examples 1 and 2), so that while sensitization was observed in the latter (Comparative Examples 1 and 2), no sensitization was observed in the former (Examples 1 and 2).

Moreover, with the asenapine-containing patches obtained in Comparative Examples 3 to 10, whose adhesive agents had different compositions from those of the asenapine-containing patches according to the present invention obtained in Examples 1 and 2, sensitization caused by the drug against the skin was not reduced and sensitization was observed even in the cases where the average cumulative amount of skin permeation of asenapine (24 hours in terms of free form) was in the range of the present invention (Comparative Examples 3, 5, and 6 to 9).

According to the present invention, it is possible, for a patch containing asenapine as a drug, to sufficiently reduce sensitization caused by the drug against the skin.

What is claimed is:

1. A method for reducing skin sensitization of an asenapine-containing patch comprising a backing layer and an adhesive agent layer, the method comprising:
using an adhesive agent for preparing the adhesive agent layer, the adhesive agent containing free asenapine in a range of 2 to 5% by mass, styrene-isoprene-styrene block copolymer (SIS) in a range of 7 to 18% by mass, polyisobutylene (PIB) in a range of 0.5 to 10% by mass, alicyclic saturated hydrocarbon resin in a range of 30 to 70% by mass, and liquid paraffin in a range of 5 to 10% by mass, relative to a total amount of the adhesive agent; and
preparing the asenapine-containing patch whose average cumulative amount of skin permeation of asenapine (24 hours) after applying the asenapine-containing patch to skin of a dorsal scapular region of a 5-week-old Hartley white female guinea pig and holding the asenapine-containing patch with an occlusive dressing for 24 hours is in a range of 90 to 300 μg/cm² in terms of free form.

2. The method for reducing skin sensitization according to claim 1, wherein an amount of the adhesive agent layer after drying is in a range of 80 to 400 g/m².

3. The method for reducing skin sensitization according to claim 1, wherein the adhesive agent further contains isopropyl palmitate in a range of 1 to 20% by mass.

4. The method for reducing skin sensitization according to claim 1, wherein a mass ratio between the SIS and the PIB (SIS:PIB) is in a range of 4:1 to 3:2.

5. The method for reducing skin sensitization according to claim 1, wherein a mass ratio between the free asenapine and a total amount of the SIS and the PIB (free asenapine:(SIS+PIB)) is in a range of 1:3 to 1:14.

6. The method for reducing skin sensitization according to claim 1, wherein a mass ratio between a total amount of the SIS and the PIB, and the alicyclic saturated hydrocarbon resin ((SIS+PIB):alicyclic saturated hydrocarbon resin) is in a range of 1:1.1 to 1:5.

7. The method for reducing skin sensitization according to claim 1, wherein amass ratio between the free asenapine and the alicyclic saturated hydrocarbon resin (free asenapine:alicyclic saturated hydrocarbon resin) is in a range of 1:6 to 1:30.

8. The method for reducing skin sensitization according to claim 1, wherein a mass ratio between a total amount of the SIS and the PIB, and the liquid paraffin ((SIS+PIB):liquid paraffin) is in a range of 4:1 to 1:1.

9. The method for reducing skin sensitization according to claim 1, wherein amass ratio between the free asenapine and the liquid paraffin (free asenapine:liquid paraffin) is in a range of 1:1 to 1:5.

10. The method for reducing skin sensitization according to claim 3, wherein amass ratio between the free asenapine and the isopropyl palmitate (free asenapine:isopropyl palmitate) is in a range of 1:1 to 1:5.
